(19) United States
(12) Patent Application Publication
(10) Pub. No.: US 2008/0039433 A1
(43) Pub. Date: Feb. 14, 2008

(54) Stabilized Tetracycline Compositions

(76) Inventors: Alexander D. Smith, Cary, NC (US); Joe Cobb, Greenville, NC (US); John Bettis, Greenville, NC (US); William C. Stagner, Apex, NC (US); Brad Gold, Greenville, NC (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/763,923

(22) Filed: Jun. 15, 2007

Related U.S. Application Data
(60) Provisional application No. 60/814,255, filed on Jun. 16, 2006.
(60) Provisional application No. 60/813,925, filed on Jun. 15, 2006.

(51) Int. Cl. A61K 31/165; A61P 29/00

(52) U.S. Cl. 514/152

(57) ABSTRACT

The invention provides a package that comprises a first rapidly disintegrating dosage form comprising tetracycline, and a second rapidly disintegrating dosage form that comprises a buffer. The invention also provides methods for treating or preventing mucositis, comprising mixing the dosage forms of the package in an aqueous medium to form a solution or suspension, and administering the solution or suspension topically to the oral cavity of a patient. The invention further provides an aqueous formulation comprising the first and second dosage forms in an aqueous medium.

This application claims priority from U.S. Provisional Application No. 60/813,925, filed Jun. 15, 2006, and U.S. Provisional Application No. 60/814,255, filed Jun. 16, 2006; both of which are incorporated by reference.

### BACKGROUND OF THE INVENTION

1. Field of the Invention

The invention relates to packages comprising a first rapidly disintegrating solid dosage form containing a tetracycline, and a second rapidly disintegrating a solid dosage form containing a buffer or a base. The two dosage forms can be added to an aqueous medium, where they rapidly dissolve, thereby forming a solution that is useful for treating or preventing mucositis when administered topically to the oral cavity.

2. Description of the Related Art

Tetracycline is a broad-spectrum antibiotic from the certain streptomyces species. Tetracycline is typically used to treat bacterial infections, such as infections of the skin, respiratory tract, genital and urinary systems, and stomach. Tetracycline is also used to treat Lyme disease. Tetracycline works by preventing the growth and spread of bacteria.

Tetracycline antibiotics degrade rapidly to form epitetracycline, anhydrotetracycline, epianhydrotetracycline, and other degradation products. Once degraded, tetracycline has no therapeutic value, since the degradation products have no therapeutically useful activity. Tetracycline degradation begins as soon as it is in solution, and continues until reaching equilibrium of tetracycline and epimer concentrations. The equilibrium point is temperature and pH dependent, with more epimer being formed at higher temperatures and lower pH. After equilibrium is established, oxidation and other side reactions cause further degradation. Thus, tetracycline products have a very limited existence in aqueous environments. Consequently, tetracycline cannot be stored in solution for extended periods of time.

Therefore, there is a need for tetracycline formulations that remain therapeutically effective during long term storage.

### SUMMARY OF THE INVENTION

In a broad aspect, the invention provides a pharmaceutical composition comprising: (a) a first dosage form comprising a therapeutically effective amount of a tetracycline; and (b) a second dosage form comprising a base or buffer, the second dosage form being physically separated from the first dosage form. In certain aspects, the first dosage form comprises meclocycline sulfosalicylate, and the second dosage form comprises tromethamine. When simultaneously or nearly simultaneously placed in an aqueous medium, the dosage forms rapidly disintegrate, and the tetracycline rapidly dissolves in the resulting mixture, thereby forming a solution of the tetracycline. The resulting solution may also contain water insoluble particulate or granular material that was released from the tablets upon disintegration.

The invention also provides methods for treating and/or preventing mucositis comprising mixing the dosage forms in an aqueous medium to form a solution or suspension, and administering the solution or suspension to a patient's oral cavity.

The invention further provides an aqueous formulation comprising (a) a solution phase comprising a tetracycline (such as meclocycline or meclocycline sulfosalicylate) and a buffer dissolved in water, and (b) a solid phase present or suspended in the solution, the solid phase comprising water insoluble material, where the water insoluble material, preferably in a particulate or granular form, comprises tablet binder, carrier, adjuvant, excipient, diluent, disintegrant, glidant, or a combination thereof.

Specific preferred embodiments of the invention will become evident from the following more detailed description of certain preferred embodiments and the claims.

### DETAILED DESCRIPTION OF THE INVENTION

In a particular embodiment, the invention provides a package that comprises a rapidly disintegrating dosage form comprising a tetracycline, preferably meclocycline, and most preferably meclocycline sulfosalicylate. The package further comprises a second rapidly disintegrating dosage form comprising a buffer.

As used herein, “rapidly” generally means that the dosage form dissolves or disintegrates within a short time, for example within about one to five minutes, preferably about one minute, when placed in an aqueous medium. Preferably, the aqueous medium is water. Shorter dissolution times, e.g., about 15, 30 or 45 seconds, are also within the scope of the invention.

Preferred buffers include for use in the second dosage form include tris(hydroxymethyl)aminomethane (tromethamine); monobasic phosphate salts such as monobasic sodium phosphate and monobasic potassium phosphate; dibasic phosphate salts such as dibasic sodium phosphate dibasic potassium phosphate, and dibasic sodium/potassium phosphate; tribasic phosphate salts, such as sodium phosphate tribasic, potassium phosphate tribasic, and tribasic sodium/potassium phosphate; sodium pyrophosphate; lysine; or a combination of at least two of the above.

The first dosage form typically further comprises a filler/binder/disintegrant, such as a cellulose derivative, e.g., hydroxymethylcellulose or microcrystalline cellulose; lactose (preferably lactose DT); pregelatinized starch; or corn starch; etc. A particularly preferred cellulose derivative is microcrystalline cellulose. Silicified microcrystalline cellulose is still more preferred.

The first dosage form generally also comprises a lubricant, such as magnesium stearate, stearic acid, talc, or combinations thereof. Magnesium stearate is preferred.

The first dosage form typically further comprises excipients such as microcrystalline cellulose, lactose, povidone, silicon dioxide (such as colloidal silicone dioxide NF), corn starch or pregelatinized starch. Silicon dioxide is preferred. Colloidal silicone dioxide NF is more preferred.

The first dosage form typically further comprises a coloring agent.

In one embodiment, the invention provides a package where the first dosage form further comprises at least two of silicified microcrystalline cellulose, magnesium stearate, silicone dioxide or a coloring agent.

The second dosage form typically further comprises a filler/binder/disintegrant, such as a cellulose derivative, e.g., hydroxymethylcellulose or microcrystalline cellulose; lactose (preferably lactose DT); pregelatinized starch; or corn starch; etc. A particularly preferred cellulose derivative is microcrystalline cellulose. Silicified microcrystalline cellulose is still more preferred.

The second dosage form typically comprises a disintegrant such as croscarmellose sodium, microcrystalline cellulose, crospovidone, sodium starch glycolate NF (EXPLOTAB®), or combinations thereof. Sodium starch glycolate NF and croscarmellose are preferred.

The second dosage form further typically comprises a lubricant, such as magnesium stearate, stearic acid, talc, or combinations thereof. Magnesium stearate is preferred.

In one embodiment, the invention provides a package where the second dosage form further comprises at least two of silicified microcrystalline cellulose, sodium starch glycolate NF, and magnesium stearate.

In a certain embodiment, the invention provides a package where the first dosage form comprises about 35-55 mg of a tetracycline, a pharmaceutically acceptable salt thereof, or a combination thereof; about 180-255 mg of filler/binder/disintegrant, such as silicified microcrystalline cellulose; and about 1-3 mg of a lubricant, such as magnesium stearate.

In a certain embodiment, the invention provides a package where the second dosage form comprises about 70-110 mg of a buffer, such as tromethamine, about 280-360 mg of a filler/binder/disintegrant, such as silicified microcrystalline cellulose; about 25-40 mg of a disintegrant, such as sodium starch glycolate NF, and about 2.5-4.1 mg of a lubricant, such as magnesium stearate.

In a preferred embodiment, the first dosage form comprises about 38-52 mg of a tetracycline, a pharmaceutically acceptable salt thereof, or a combination thereof; about 190-245 mg of filler/binder/disintegrant, such as silicified microcrystalline cellulose; and about 1.1-2.9 mg of a lubricant, such as magnesium stearate; and the second dosage form comprises about 80-100 mg of buffer, preferably, tromethamine; about 290-350 mg of filler/binder/disintegrant, such as silicified microcrystalline cellulose; about 27-38 mg of disintegrant, such as sodium starch glycolate NF; and about 2.7-3.9 mg of lubricant, such as magnesium stearate.

In a preferred embodiment, the first dosage form comprises about 35-55 mg of a memlocycline sulfosalicylate; about 190-230 mg of silicified microcrystalline cellulose; about 1-3 mg of magnesium stearate; and about 0.4-1 mg of colloidal silicon dioxide NF.

In another preferred embodiment, the second dosage form comprises about 80-100 mg of tromethamine; about 300-340 mg of silicified microcrystalline cellulose; about 30-45 mg of sodium starch glycolate NF; and about 3.0-3.7 mg of magnesium stearate.

In still another preferred embodiment, the first dosage form comprises about 35-55 mg of a memlocycline sulfosalicylate; about 190-230 mg of silicified microcrystalline cellulose; about 1-3 mg of magnesium stearate; and about 0.4-1 mg of colloidal silicon dioxide NF; and where the second dosage form comprises about 80-100 mg of tromethamine; about 300-340 mg of silicified microcrystalline cellulose; about 30-45 mg of sodium starch glycolate NF; and about 3.0-3.7 mg of magnesium stearate.

In certain embodiments, the invention provides methods for treating and/or preventing mucositis by administrating to a patient a formulation, comprising an effective amount of a tetracycline, administered as a mouth-rinse (mouthwash) or oral liquid.

In another embodiment, the invention provides a method for treating or preventing oral mucositis resulting from radiotherapy or chemotherapy, the method comprising administering to a patient a mouthwash solution, wherein the mouthwash solution is prepared by adding the first dosage form and the second dosage form of the package of claim 1 to an aqueous medium, and then removing the mouthwash solution.

A package can be any container that keeps the two dosage forms separate. For example, the package can be a blister pack, wherein each dosage form is contained in a separate compartment of the blister pack. Preferably, each blister pack contains a first dosage form comprising a tetracycline, and a second dosage form comprising a buffer. Preferred packages of the invention also contain instructions describing for the patient user how to use the dosage forms of the invention. The instructions detail how much aqueous medium to use, how many of each dosage form to place in the aqueous medium, how long to wait after placing the dosage form in the medium, and how to use the resulting mixture of meclocycline in solution and suspension of excipients.

The dosage form in a package of the invention can be in the form of, for example, sugar-coated tablets, film-coated tablets, multiple compressed tablets (including layered and press coated tablets), tablets for making a solution, effervescent tablets, sustained release tablets, extruded tablets, frozen tablets, hard tablets, soft tablets, fast disintegrating tablets, pellets, granules, microspheres, powder or shaped powders. The form of each dosage form is independently selected from the above.

The buffer in the package of the invention is included to adjust the pH, for example, to about 6-10, preferably about 7-9 (or more preferably about 8-9), when the first and second dosage forms are added to an aqueous medium. This pH range helps maximize the solubility and stability of the tetracycline (such as meclocycline sulfosalicylate) in the aqueous medium.

The aqueous medium can be saliva or water (preferably water) in a volume of, for example, about 5-25 ml, preferably about 10-20 ml, and most preferably about 15 ml, in which the solid dosage forms disintegrate or dissolve to form a mouth rinse.

As used herein, “water” refers to distilled water, deionized water, bottled water, tap water, and water having salts, minerals, etc., dissolved in it.

Typically the buffer or base is used in a molar excess compared to the tetracycline. Suitable molar ratios of base/buffer to tetracycline are from about 3:1 to 75:1. Preferred molar ratios are from about 7:1 to 25:1. More preferred molar ratios are from about 10:1 to 20:1. Particularly preferred molar ratios of base/buffer to tetracycline are from about 12:1 to 18:1.

The tetracycline can be in the form of a pharmaceutically acceptable salt or the free base form. The formulation may contain other agents such as a non-steroidal anti-inflammatory drug (NSAID), an inflammatory cytokine inhibitor, a mast cell inhibitor, an MMP inhibitor, an NO inhibitor, or a mixture thereof. The formulations can optionally also contain an antifungal agent to prevent fungal overgrowth due to reduction in the normal oral flora by the tetracycline or another agent.

As used herein, tetracyclines include compounds that may or may not have antibiotic activity. The tetracyclines described herein can have high or poor water solubility and can be well absorbed or poorly absorbed from the gastrointestinal tract. Solubility may be reduced by forming poorly soluble salts. Preferred tetracyclines are those that are poorly absorbed when administered orally. Compounds which have bioavailabilities of 50% or less are considered to be poorly absorbed.

Tetracyclines are defined by the following structure: 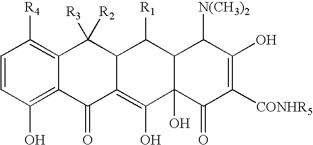
wherein R₁-R₅ may be a hydrogen atom, a halogen atom, a hydroxyl group, or any other organic composition comprising from 1-8 carbon atoms and optionally include a heteroatom such as nitrogen, oxygen, in linear, branched, or cyclic structural formats.

A wide range and diversity of embodiments within the definition of the above structure are described within Essentials of Medicinal Chemistry John Wiley and Sons, Inc., 1976, pages 512-517. Preferably R₁ and R₂ are hydrogen or a hydroxyl group; R₃ is hydrogen or a methyl group; R₄ is a hydrogen atom, a halogen, or a nitrogen containing entity; and R₅ is a hydrogen atom, or nitrogen containing ring structure. The commonly known tetracycline analogues and derivatives include the following: oxytetracycline; chlortetracycline; demeclocycline; doxycycline; minocycline; rolitetracycline; lymecycline; sancycline; tetracycline; methacycline; apicycline; clomocycline; guamecycline; meglucycline; mepyclcline; penimepicycline; pipacycline; etocycline, penimocycline, and meclocycline.

Tetracycline derivatives that can be used as described herein include tetracycline derivatives modified at positions 1 through 4 and 10 through 12, although these modifications may result in reduction in antibiotic properties, according to Mitscher, et al., J. Med. Chem. 21(5), 485-489 (1978). The configuration of the 4 carbon is important to the antibiotic properties of the tetracyclines. For the antibiotic tetracyclines, carbon 4 is in the S configuration. The 4-epimers of the tetracyclines, which have the R configuration at the 4 carbon, have significantly reduced antibiotic activity. Other such non-antibiotic tetracycline analogs include the 4-de(dimethylamino) derivatives of the tetracyclines listed in the above paragraph. Specific examples include: 6 demethyl-6-deoxy-4-dedimethylaminotetracycline; 6-demethyl-6-deoxy-4dedimethylamino-7-dimethylaminotetracycline; 6-demethyl-6-deoxy-4dedimethylamino-7-chloro-tetracycline; 4-hydroxy-4dedimethylaminotetracycline; 6a-deoxy-5-hydroxy-4dedimethylaminotetracycline; 4-dedimethylamino-5-oxytetracycline, and 4dedimethylamino-11-hydroxy-12a-deoxytetracycline. Further examples of tetracyclines with reduced antibiotic activity include 6-α-benzylthiomethylenetetracycline, 6-fluoro-6-demethyltetracycline, and 11α-chlorotetracycline.

Other tetracycline related compounds that can be used as described herein are the 9-((substituted)amido)tetracyclines. The latter include the compounds described in U.S. Pat. Nos. 5,886,175, 5,284,963, 5,328,902, 5,386,041, 5,401,729, 5,420,272, and 5,430,162.

Preferred poorly absorbed tetracyclines include compounds of the following structure: 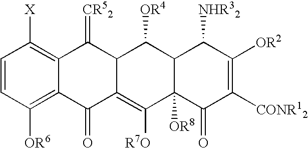
wherein R¹, R², R³, R⁴, R⁵, R⁶, R⁷, and R⁸ can be H, C1-C3 alkyl, phenyl, and aryl groups; and
wherein X is an H, alkyl, alkoxy, phenoxy, aryloxy, amino group, amide, acyl, and halo group; and pharmaceutically acceptable salts thereof.

The most preferred compound of this general structure is wherein R¹, R², R⁴, R⁵, R⁶, R⁷, and R⁸ are H; wherein R³ is CH₃; and wherein X is a chloro group. The generic name for this compound is meclocycline. The preparation of meclocycline and its analogs and derivatives are known. For example, U.S. Pat. No. 3,966,808 to Luciano discloses methods for manufacturing 6-methylenetetracyclines.

The term “pharmaceutically acceptable salt” refers to those salts of tetracyclines that are not substantially toxic at the dosage administered to achieve the desired effect and do not independently possess significant pharmacological activity. The salts included within the scope of this term are pharmaceutically acceptable acid addition salts of a suitable inorganic or organic acid. Suitable inorganic acids are, for example hydrochloric, hydrobromic, sulfuric and phosphoric acids. Suitable organic acids include carboxylic acids, such as acetic, propionic, glycolic, lactic, pyruvic, malonic, succinic, fumaric, malic, tartaric, citric, cyclamic, ascorbic, maleic, hydroxymaleic, dihydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acid, sulfonic acids, such as methanesulfonic, sulfosalicylate, ethanesulfonic and .beta.-hydroxyethanesulfonic acid. Sulfosalicylate is a preferred salt. In addition, pharmaceutically acceptable salts include those salts of tetracyclines formed with inorganic and organic bases, such as those of alkali metals, for example sodium, potassium and lithium, alkaline earth metals, for example calcium and magnesium, light metals of group IIIA, for example aluminum, organic amines, for example primary, secondary or tertiary amines, such as cyclohexylamine, ethylamine, pyridine, methylaminoethanol and piperazine. The salts are prepared by conventional means by one of ordinary skill in the art as, for example, by treating a tetracycline with an appropriate acid or base. Such salts can exist in either a hydrated or substantially anhydrous form.

An aqueous formulation comprising (a) a solution phase, and (b) a water-insoluble phase present or suspended in the solution phase, where, at least one tetracycline, tetracycline salt, or a combination thereof, and at least one buffer are mostly (preferably completely) dissolved in the solution phase, where the solution phase is an aqueous medium, and where the solid phase comprises water insoluble solid material.

In another aspect, the invention provides an aqueous formulation comprising (a) a solution phase comprising a tetracycline (such as meclocycline or meclocycline sulfosalicylate) and a buffer dissolved in water, and (b) a solid phase present or suspended in the solution, the solid phase comprising water insoluble material, where the water insoluble material, preferably in a particulate or granular form, comprises tablet binder, carrier, adjuvant, excipient, diluent, disintegrant, glidant, or a combination thereof.

In one embodiment, the buffer in the solution phase of the aqueous formulation is tris(hydroxymethyl)aminomethane (tromethamine); a monobasic phosphate salt, such as monobasic sodium phosphate or monobasic potassium phosphate; dibasic phosphate salts, such as dibasic sodium phosphate, dibasic potassium phosphate, or dibasic sodium/potassium phosphate; tribasic phosphate salts, such as sodium phosphate tribasic, potassium phosphate tribasic, or tribasic sodium/potassium phosphate; sodium pyrophosphate; lysine; or a combination thereof. Tromethamine is a preferred buffer.

In another embodiment, the water insoluble material comprises a filler/binder/disintegrant, such as a cellulose derivative, e.g., hydroxymethylcellulose or microcrystalline cellulose; etc. A particularly preferred cellulose derivative is microcrystalline cellulose. Lactose is also a preferred filler/binder/disintegrant.

In still another embodiment, the water insoluble material comprises a disintegrant, such as croscarmellose sodium, microcrystalline cellulose, crospovidone, sodium starch glycolate NF, or combinations thereof. Sodium starch glycolate and croscarmellose sodium are preferred disintegrants.

In still another embodiment, the water insoluble material comprises a lubricant, such as magnesium stearate, stearic acid, talc, or combinations thereof. Magnesium stearate is preferred.

In still another embodiment, the water insoluble material comprises microcrystalline cellulose (such as silicified microcrystalline cellulose), a disintegrant (such as sodium starch glycolate NF or croscarmellose sodium NF), and a lubricant (such as magnesium stearate).

In one embodiment, the formulation further comprises a filler/binder/disintegrant, such as lactose. When present, the lactose dissolves in the water.

The aqueous formulation is used shortly after it is prepared, and preferably within about 5 minutes of its preparation.

The aqueous formulation may further comprise one or more flavoring agents, coloring agents, or combinations thereof.

To make the aqueous formulation, the tablets are added to about 9-20 ml of water (preferably about 15 mL), and then shaken for approximately 30 seconds. The tablets may be simultaneously added to the water, or they may be sequentially added to the water. The tablets will disintegrate within about 5-20 seconds (preferably 6-15 seconds, more preferably about 8-12 seconds, and still more preferably about 10 seconds). Preferably, at least about 90% of the tetracycline, the buffer, and the lactose (if present) will dissolve within the approximately 30 seconds, after which the water insoluble material will still be visibly present. The water insoluble material may comprise particulates; therefore, the resulting aqueous formulation may appear cloudy.

If desired, the aqueous formulation may be filtered before use.

After making the aqueous formulation, the patient rinses her mouth with the solution for approximately 30 seconds. The patient may also use the solution as a gargle, in order to treat the back of the oral cavity, i.e., the upper region of the throat.

Preferably, the patient will not rinse her mouth for at least about 30 minutes after dosing. Additionally, it is preferable that the patient not eat or drink anything for approximately 30 before and approximately 30 minutes after rinsing with the aqueous formulation.

Active agents other than tetracycline can also be used in dosage forms of the invention to aid in the treatment or prevention of mucositis. These agents can be inflammatory cytokine inhibitors, and/or mast cell inhibitors and/or NO inhibitors that reduce and inhibit mucositis.

Agents that inhibit the function of the mast cells or the action of the mediators released by mast cells can be used to treat and prevent mucositis. Mast cell inhibitors are chemical or biological agents that suppress or inhibit the function of mast cells, or the mediators released by mast cells. For example, mast cell inhibitors can inhibit degranulation, thereby preventing the release of mediators into the extracellular space.

Examples of mast cell degranulation inhibitors include picetannol, benzamidines, tenidap, tiacrilast, disodium cromoglycate, lodoxamide ethyl, and lodoxamide tromethamine. Other agents that inhibit mediator release include staurosporine and CGP 41251. Examples of mast cell mediator inhibitors include agents that block the release or secretion of histamine, such as FK-506 and quercetin; antihistamines such as diphenhydramine; and theophylline. Other mast cell inhibitors include serine protease inhibitors, such as alpha-1-protease inhibitor; metalloprotease inhibitors; lisofylline; benzamidine; amiloride; and bis-amidines such as pentamidine and bis(5-amidino-2-benzimidazolyl)methane.

Inflammatory cytokine inhibitors are chemical or biological agents that suppress or inhibit inflammatory cytokines. Such inhibitors include pyridinyl imidazoles, bicyclic imidazoles, oxpentifylline, thalidomide and gabexate mesilate.

Anti-inflammatory agents can be used in combination with inflammatory cytokine and/or mast cell inhibitors to treat and prevent mucositis. Examples of anti-inflammatory agents that can be used include the non-steroidal anti-inflammatory drugs (NSAIDs) flurbiprofen, ibuprofen, ketoprofen, sulindac, and diclofenac. When NSAIDs are administered, anti-ulcer agents such as ebrotidine can be administered, e.g., to help protect against gastric mucosal damage. Other anti-inflammatory agents that can be used include misoprostil; methylxanthine derivatives, such as caffeine, lisofylline, or pentoxyfylline; benzydamine; naprosin; mediprin; and aspirin.

Another important class of anti-inflammatory agents includes cyclooxygenase (COX) inhibitors, particularly COX-2 inhibitors. COX-2 inhibitors that can be used include celecoxib, nimesulide, meloxicam, piroxicam, flosulide, etodolac, nabumetone, and 1-[(4methylsulfonyl)phenyl]-3-triflu-oromethyl-5-[(4-fluoro)phenyl]pyrazole. Other useful anti-inflammatory agents include dual cyclooxygenase/lipoxygenase inhibitors, such as 2-acetylthiophene-2-thiazolylhydrazone, and leukotriene formation inhibitors, such as piriprost.

MMP inhibitors include both the antibacterial tetracyclines such as tetracycline HCl, minocycline and doxyocycline, as well as non-antibacterial tetracyclines.

Nitric oxide (NO) inhibitors can be any type. Preferable NO inhibitors can be aminoguanidine, guanidine or a mixture thereof.

The administration of anti-microbial agents in combination with the agents described above can result in an even more effective method for treating and preventing mucositis. Examples of antimicrobial agents that can be used include agents with activity against gram positive and gram negative organisms. Specific drugs include tetracycline HCl, amoxicillin, gentamicin, and chlorhexidine.

Other agents that may be used to treat or prevent mucositis in combination with tetracyclines include the nuclear transcription factor kappa-B (NF-B) activation inhibitors capsaicin and resiniferatoxin.

Other medicinal agents may be added to dosage forms of the invention for purposes of alleviating other undesirable conditions in the mouth. Such agents may include, for example, local anesthetics, antibacterial agents, and emollients, as well as anti-fungal agents.

In certain embodiments, one of the solid dosage forms in a package of the invention contains preferably about 0.1-100.0 mg, more preferably, about 1 to 75, or about 20-80, or about 30-60 mg, and most preferably about 30, 45, or 60 mg, of the tetracycline, as a salt, preferably the sulfosalicylate. In a particular embodiment, the tetracycline is meclocycline sulfosalicylate. Preferred embodiments of the invention contain about 47 mg of meclocycline sulfosalicylate in each tablet (first dosage form), providing about 30 mg of meclocycline per tablet. In other preferred embodiments of the invention contain about 94 mg of meclocycline sulfosalicylate in each tablet (first dosage form), providing about 60 mg of meclocycline per tablet. In other embodiments, one of the solid dosage forms in a package of the invention contains preferably about 0.1-100.0 mg, more preferably about 50 to 100 mg, and most preferably about 90 mg of a buffer, such as tromethamine. Other buffers/bases can suitably be used in the invention. Suitable buffers and bases are preferably inorganic bases; representative examples include bases such as sodium phosphate tribasic, sodium phosphate dibasic, sodium bicarbonate, sodium hydroxide, etc.

The solid dosage forms of a package of the invention can be added to a liquid vehicle to produce a mouth rinse. The mouth rinse is preferably prepared by the patient immediately prior to administration.

The mouth rinse compositions are administered to the oral cavity, held and swished around in the mouth, and then swallowed or spit out. The liquid vehicle is preferably water. Other components may be present in the vehicle as described below.

Liquid formulations may contain additional components to improve the effectiveness of the product. For example, one or more components can be added to increase viscosity to provide improved retention on the surfaces of the oral cavity. Suitable viscosity increasing agents include carboxyalkyl, hydroxyalkyl, and hydroxyalkyl alkyl celluloses, xanthan gum, carageenan, alginates, pectins, guar gum, polyvinylpyrolidone, gellan gums, and gelatin. High viscosity formulations may cause nausea in chemotherapy and radiation patients and are therefore not preferred. Gelatin or its derivatives are preferred as viscosity modifying agents. Gellan gums are also preferred modifying agents since aqueous solutions or suspensions containing certain gellan gums may be prepared so that they will experience an increase in viscosity upon contact with electrolytes. Saliva contains electrolytes that will interact with such a gellan containing solution so as to increase their viscosity. The increased viscosity will promote retention of the solutions in the oral cavity and provide greater effectiveness due to increased contact time with the affected tissues.

In order to improve the patient acceptability, appropriate coloring and/or flavoring material can be added to a liquid vehicle before or after contact with the dosage forms of a package of the invention. Alternatively, the coloring and/or flavoring material can be added to one or both of the tablets. Any pharmaceutically acceptable coloring or flavoring material may be used. Flavorings used in the mouth rinse art such as peppermint, citrus flavorings, berry flavorings, custard, vanilla, cinnamon, and sweeteners, either natural or artificial, may be used. Flavorings that are known to increase salivary electrolyte concentrations may be added to increase the magnitude of the viscosity change.

Pharmaceutically acceptable fillers and excipients can be used to formulate the tetracyclines and the other optional agents described herein into solid dosage forms. Suitable solid dosage forms include powders or tablets that are designed for constitution as solutions by dissolution or suspension in a liquid vehicle. In one preferred embodiment, the solid dosage form is a tablet.

For convenience of use, solid dosage forms in a package of the invention are preferably rapidly disintegrating. Technologies to produce rapidly disintegrating solids are well known in the art. These include spray-drying, use of disintegrants and water insoluble components, freeze-drying, particle size reduction and optimizing the pH of the dissolution medium.

Additional excipients generally known in the art can be used to formulate the tetracyclines and optional agents into a suitable dosage form (see, for example, Encyclopedia of Controlled Drug Delivery, Edith Mathiowitz, Ed., John Wiley & Sons, Inc., New York, 1999; and U.S. Pat. No. 5,558,880, the teachings of which and references cited therein are incorporated herewith by reference). For example, for a solid dosage form such as tablet prepared by a freeze-drying process, sugars such as lactose and/or mannitol or the derivatives thereof can be used in the formulation.

Various solid dosage forms, the materials making the solid dosage forms, and methods for making the solid dosage forms have been described. For example, U.S. Pat. Nos. 6,316,027; 5,648,093; and 4,754,597 disclose fast disintegrating dosage forms of a drug and the process of making the dosage forms. U.S. Pat. Nos. 6,156,339; 5,837,287; 5,827,541 describe methods for the preparation of solid rapidly disintegrating dosage forms of a drug.

Various forms of blister pack and the method of making the pack or the blister pack form of a drug has been described in, for example, U.S. Pat. Nos. 5,729,958; 5,046,618; 5,343,672; and 5,358,118. U.S. Pat. No. 5,631,023 discloses rapidly dispersing pharmaceutical tablets of a drug. U.S. Pat. No. 5,558,880 discloses a fast disintegrating, solid dosage form formed of a matrix containing gelatin, pectin and/or soy fiber protein. U.S. Pat. No. 5,188,825 describes using an ion exchange resin to bond a water soluble active agent so as to form a substantially water insoluble complex. The teachings of these U.S. patents are incorporated herein by reference.

Various methods for making rapidly disintegrating solid dosage forms of a drug have been described in, for example, U.S. Pat. Nos. 6,316,027; 5,648,093; 4,754,597; 6,156,339; 5,837,287; 5,827,541; 5,729,958; 5,046,618; 5,343,672; 5,358,118; 5,631,023; 5,558,880; 5,188,825; 6,221,392; 6,024,981; and 5,576,014, the teachings of which are incorporated herein by reference.

Methods of using the mixture (in preferred embodiments, meclocycline is in solution and the various excipients are in suspension) that results from mixing the dosage forms from a package of the invention in an aqueous medium as disclosed herein generally involve applying the formulations topically to mucosal surfaces of the oral cavity and gastrointestinal tract. The method includes the step of mixing the solid dosage forms from a package of the invention in an aqueous medium to form a solution or suspension, and administering to a patient an effective amount of a solution or suspension. The solution is administered as, for example, a mouth rinse. In one embodiment, the method is for treating or preventing oral mucositis resulting from radiation or chemotherapy for cancer. In preferred aspects of the invention, after combination in the aqueous medium, the active ingredient is in solution, and the tablet excipients are in suspension.

In one embodiment, one to eight applications per day beginning 24 hours before chemotherapy or radiation until conclusion of treatment are made. A typical volume of a mouthwash would be between 5-15 ml, preferably about 10 ml. Therapy can continue for as long as the patient is receiving radiation or chemotherapy.

In a preferred aspect, the tablets are used to prepare an aqueous mouth rinse composition which is immediately, i.e., within about 5 minutes of preparation, used to rinse the oral cavity. More preferably, the composition is used to rinse the oral cavity within 3 minutes of adding the composition to the water. Still, more preferably, the composition is used to rinse the oral cavity within 1 minute of adding the composition to the water. To prepare the aqueous mouth rinse, the tablets are added to a predetermined amount, e.g., 5 ml, 10 ml, 15 ml, 20 ml, or 25 ml, or water, typically tap water, after which the water/tablet mixture may be mixed by stirring or shaking to disintegrate and dissolve the tablet components. In preferred aspects of the invention, the tetracycline (or salt thereof) and the buffer will dissolve in the water, while other components, in particular the disintegrants will be insoluble. In addition to the tetracycline and buffer dissolving, in preferred aspects, the lactose (if present) will also dissolve in the water.

Unless otherwise required by context, singular terms as used herein shall include pluralities and plural terms as used herein shall include the singular.

The Examples that follow are merely illustrative of specific embodiments of the invention, and are not to be taken as limiting the invention, which is defined by the appended claims.

### EXAMPLES

### Example 1

Separate tablets containing 30 mg Meclocycline and 90 mg Tris(tromethamine) are prepared to contain the components listed in Tables 1 and 2 respectively. Pairs of the Meclocycline and tromethamine tablets are packaged in foil/foil blister packs.

TABLE 1

| | | Amount | Amount |
|---|---|---|---|
| | Weight | per | per |
| | Percent | Tablet | Batch |
| INGREDIENTS | (%) | (mg) | (g) |
| Meclocycline Sulfosalicylate | 18.15 | 47.20* | 236.00 |
| Silicified Microcrystalline | 42.18 | 109.7 | 548.50 |
| Cellulose (PROSOLV SMSS 90, | | | |
| commercially available from | | | |
| JRS Pharma LP, Patterson, NY)) | | | |
| Lactose Monohydrate, NF | 30.92 | 80.39 | 401.90 |
| (Foremost 316 Fast Flo) | | | |
| Croscarmellose Sodium, NF | 7.500 | 19.50 | 97.50 |
| (Ac-Di-Sol SD-711) | | | |
| Magnesium Stearate, NF | 0.7500 | 1.950 | 9.75 |
| FD&C Yellow #6 Aluminum Lake | 0.4000 | 1.040 | 5.200 |
| (17-19%) | | | |
| FD&C Yellow #6 Dye | 0.1000 | 0.2600 | 1.300 |
| Total | 100.0 | 260.0 | 1300.15 |

*Added as Meclocycline Sulfosalicylate; correction factor applied to Sulfosalicylate salt based on assay of drug substance.

TABLE 2

| | | AMOUNT | AMOUNT |
|---|---|---|---|
| | WEIGHT | PER | PER |
| | PERCENT | TABLET | BATCH |
| INGREDIENTS | (%) | (mg) | (g) |
| Tromethamine, USP | 20.00 | 90.00 | 500.0* |
| (Tris) | | | |
| Silicified Microcrystalline | 41.50 | 186.8 | 934.0 |
| Cellulose (PROSOLV SMSS 90) | | | |
| Lactose Monohydrate, NF | 30.25 | 136.1 | 680.5 |
| (Foremost 316 Fast Flo) | | | |
| Croscarmellose Sodium, NF | 7.500 | 33.75 | 168.8 |
| (Ac-Di-Sol SD-711) | | | |
| Magnesium Stearate, NF | 0.7500 | 3.375 | 16.88 |
| Total | 100.0 | 450.0 | 2300.18 |

### Example 2

Tablets containing 30 mg Meclocycline are prepared to contain the following components. Buffer tablets for use with the 30 mg Meclocycline tablets of this example are prepared as shown above for Example 1 to contain 90 mg of a buffer, Tromethamine. Pairs of the Meclocycline and tromethamine tablets are packaged in foil/foil blister packs.

| | Tablet A | Tablet B | Tablet C |
|---|---|---|---|
| Ingredient | (mg) | (mg) | (mg) |
| Meclocycline Sulfosalicylate | 30.00 | 30.00 | 30.00 |
| ProSolv ® 90 SMCC | 84.00 | 84.00 | 84.00 |
| Lactose 316 | 105.6 | 105.0 | 105.0 |
| Explotab ® (Sodium Starch | 18.00 | 18.00 | — |
| Glycolate, commercially | | | |
| available from JRS Pharma | | | |
| LP, Patterson, NY) | | | |
| Croscarmellose Sodium, NF | — | — | 18.00 |
| Magnesium Stearate | 1.800 | 1.800 | 1.800 |
| Yellow #6 Aluminum Lake | 0.4800 | 0.9600 | 0.9600 |
| (17-19%) | | | |
| Yellow #6 Dye | 0.1200 | 0.2400 | 0.2400 |
| TOTAL | 240.0 | 240.0 | 240.0 |

— = not present

### Example 3

Tablets containing 60 mg Meclocycline are prepared to contain the following components. Buffer tablets for use with the 60 mg Meclocycline tablets are prepared as shown above for Example 1 to contain 180 mg of a buffer, Tromethamine. Pairs of the Meclocycline and tromethamine tablets are packaged in blister packs.

| | Tablet A | Tablet B | Tablet C |
|---|---|---|---|
| Ingredient | (mg) | (mg) | (mg) |
| Meclocycline Sulfosalicylate | 60.00 | 60.00 | 60.00 |
| ProSolv ® 90 SMCC | 168.00 | 168.00 | 168.00 |
| Lactose 316 | 211.2 | 210.0 | 210.0 |
| Explotab ® (Sodium Starch | 36.00 | 36.00 | — |
| Glycolate, commercially | | | |
| available from JRS Pharma | | | |
| LP, Patterson, NY) | | | |
| Croscarmellose Sodium, NF | — | — | 36.00 |
| Magnesium Stearate | 3.600 | 3.600 | 3.600 |
| Yellow #6 Aluminum Lake | 0.9600 | 1.9200 | 1.9200 |
| (17-19%) | | | |
| Yellow #6 Dye | 0.2400 | .04800 | .04800 |
| TOTAL | 480.0 | 480.0 | 480.0 |

— = not present

### Example 4

Tablets containing 45 mg of micronized Meclocycline Sulfosalicylate are prepared to contain the following components. Buffer tablets for use with the 45 mg Meclocycline tablets are prepared as shown below and contain 90 mg of a buffer, Tromethamine. Pairs of the Meclocycline and tromethamine tablets are packaged in foil/foil blister packs.

TABLE 1

| | Amount | |
|---|---|---|
| | per | Amount |
| | Tablet | per Batch |
| INGREDIENTS | (mg) | (g) |
| Micronized Meclocycline | 45.00* | 1350 |
| Sulfosalicylate | | |
| Silicified Microcrystalline | 212.1 | 6363 |
| Cellulose | | |
| (PROSOLV SMSS 90, commercially | | |
| available from JRS Pharma LP, | | |
| Patterson, NY)) | | |
| Magnesium Stearate, NF and EP | 1.950 | 58.50 |
| (Non-bovine Hyqual) | | |
| Colloidal Silicon Dioxide NF | 0.6500 | 19.50 |
| (Cab-O-Sil ® MSP) | | |
| FD&C Yellow #6 Powder | 0.2600 | 7.800 |
| Total | 260.0 | 7798.8 |

*correction factor applied to Sulfosalicylate salt based on assay of drug substance.
Correction factor to convert free-base to salt = 1.50 from analytical certificate of analysis.
Amount of Micronized Meclocycline Sulfosalicylate per tablet = 30.00 mg Meclocycline free-base * 1.50 = 45.00 mg Micronized Meclocycline Sulfosalicylate.

TABLE 2

| | | AMOUNT | AMOUNT |
|---|---|---|---|
| | WEIGHT | PER | PER |
| | PERCENT | TABLET | BATCH |
| INGREDIENTS | (%) | (mg) | (kg) |
| Tromethamine, USP (Tris) | 20.00 | 90.00 | 2.700 |
| | | | (3.000) * |
| Silicified Microcrystalline | 71.75 | 322.9 | 9.687 |
| Cellulose (PROSOLV SMCC ® | | | |
| 90) | | | |
| Sodium Starch Glycolate, NF | 7.500 | 33.75 | 1.013 |
| Magnesium Stearate, NF and EP | 0.7500 | 3.375 | 0.1013 |
| (Non-bovine Hyqual) | | | |
| Total | 100.0 | 450.0 | 13.50 |

* an overage of Tromethamine will be dispensed to Account for Milling.

It should be understood that the foregoing disclosure emphasizes certain specific embodiments of the invention and that all modifications or alternatives equivalent thereto are within the spirit and scope of the invention as set forth in the appended claims.

We claim:

1. A pharmaceutical composition comprising:
(a) a first dosage form comprising an effective amount of a tetracycline, a pharmaceutically acceptable salt thereof, or combinations thereof; and
(b) a second dosage form comprising a buffer or a base, the second dosage form being physically separated from the first dosage form.

2. The pharmaceutical composition of claim 1, wherein the base or buffer is tromethamine, sodium phosphate tribasic, sodium phosphate dibasic, sodium bicarbonate, or sodium hydroxide.

3. The pharmaceutical composition of claim 2, wherein the base or buffer is tromethamine and the package comprises about 90 mg of tromethamine in the second dosage form.

4. The pharmaceutical composition of claim 1, wherein the tetracycline is meclocycline sulfosalicylate, which is present in the amount of 20-80 mg.

5. The pharmaceutical composition of claim 1, where the first dosage form comprises about 35-55 mg of a tetracycline, a pharmaceutically acceptable salt thereof, or a combination thereof; about 180-255 mg of silicified microcrystalline cellulose; and about 1-3 mg of magnesium stearate.

6. The pharmaceutical composition of claim 1, where the second dosage form comprises about 70-110 mg of tromethamine; about 280-360 mg of silicified microcrystalline cellulose; about 25-40 mg of sodium starch glycolate, NF; and about 2.5-4.1 mg of magnesium stearate.

7. The pharmaceutical composition of claim 1, wherein the first and second dosage forms are independently selected from the group consisting of sugar-coated tablets, film-coated tablets, effervescent tablets, frozen tablets, hard tablets, and soft tablets.

8. A method for preparing an aqueous tetracycline formulation, the method comprising:
(a) providing the package of claim 1; and
(b) adding the first and the second dosage forms to an aqueous medium.

9. The method of claim 8, wherein the first dosage form rapidly disintegrates in the aqueous medium forming a mixture comprising the tetracycline in solution.

10. The method of claim 8, wherein the aqueous medium is water.

11. The method of claim 8, wherein the buffer in the second dosage form raises the pH of the aqueous medium to about 8 to 9.

12. The method of claim 8, wherein the buffer in the second dosage form is tromethamine.

13. The method of claim 8, wherein the tetracycline in the first dosage form is meclocycline sulfosalicylate.

14. A pharmaceutical composition for treating or preventing mucositis comprising an effective amount of a tetracycline and a pharmaceutically acceptable carrier, wherein the composition is formed by mixing the first dosage form and the second dosage form of the composition of claim 1 in an aqueous solution.

15. The pharmaceutical composition of claim 14, wherein the buffer in the second dosage form is tromethamine.

16. The pharmaceutical composition of claim 14, wherein the tetracycline in the first dosage form is meclocycline sulfosalicylate.

17. The pharmaceutical composition of claim 14 formulated as a mouthwash for topical administration to the mucosa of the oral cavity and gastrointestinal tract.

18. A method for treating or preventing oral mucositis resulting from radiotherapy or chemotherapy, the method comprising administering to a patient a mouthwash solution, wherein the mouthwash solution is prepared by adding the first dosage form and the second dosage form of the composition of claim 1 to an aqueous medium, and then removing the mouthwash solution.

19. The method of claim 18, wherein the buffer is tromethamine.

20. The method of claim 18, wherein the tetracycline is meclocycline sulfosalicylate.

21. An aqueous formulation comprising (a) a solution phase, and (b) a water-insoluble phase present or suspended in the solution phase, where, at least one tetracycline, tetracycline salt, or a combination thereof, and at least one buffer are dissolved in the solution phase, where the solution phase is an aqueous medium, and where the solid phase comprises water insoluble solid material.

22. A packaged pharmaceutical composition comprising a package comprising:
(a) a first dosage form comprising an effective amount of a tetracycline, a pharmaceutically acceptable salt thereof, or combinations thereof; and
(b) a second dosage form comprising a buffer or a base, the second dosage form being physically separated from the first dosage form.

23. A packaged pharmaceutical composition according to claim 22, further comprising instructions for using the dosage forms.

24. The composition of claim 1, where the first dosage form comprises about 35-55 mg of a meclocycline sulfosalicylate; about 190-230 mg of silicified microcrystalline cellulose; about 1-3 mg of magnesium stearate; and about 0.4-1 mg of colloidal silicon dioxide NF.

25. The composition of claim 1, where the second dosage form comprises about 80-100 mg of tromethamine; about 300-340 mg of silicified microcrystalline cellulose; about 30-45 mg of sodium starch glycolate NF; and about 3.0-3.7 mg of magnesium stearate.

26. The composition of claim 1, where the first dosage form comprises about 35-55 mg of a meclocycline sulfosalicylate; about 190-230 mg of silicified microcrystalline cellulose; about 1-3 mg of magnesium stearate; and about 0.4-1 mg of colloidal silicon dioxide NF; and where the second dosage form comprises about 80-100 mg of tromethamine; about 300-340 mg of silicified microcrystalline cellulose; about 30-45 mg of sodium starch glycolate NF; and about 3.0-3.7 mg of magnesium stearate.

27. The packaged pharmaceutical composition of claim 26, wherein the base or buffer is tromethamine, sodium phosphate tribasic, sodium phosphate dibasic, sodium bicarbonate, or sodium hydroxide.

28. The packaged pharmaceutical composition of claim 27, wherein the base or buffer is tromethamine and the package comprises about 90 mg of tromethamine in the second dosage form.

29. The packaged pharmaceutical composition of claim 26, wherein the tetracycline is meclocycline sulfosalicylate, which is present in the amount of 20-80 mg.

30. The packaged pharmaceutical composition of claim 26, where the first dosage form comprises about 35-55 mg of a tetracycline, a pharmaceutically acceptable salt thereof, or a combination thereof; about 180-255 mg of silicified microcrystalline cellulose; and about 1-3 mg of magnesium stearate.

31. The packaged pharmaceutical composition of claim 26, where the second dosage form comprises about 70-110 mg of tromethamine; about 280-360 mg of silicified microcrystalline cellulose; about 25-40 mg of sodium starch glycolate, NF; and about 2.5-4.1 mg of magnesium stearate.

32. The packaged pharmaceutical composition of claim 26, wherein the first and second dosage forms are independently selected from the group consisting of sugar-coated tablets, film-coated tablets, effervescent tablets, frozen tablets, hard tablets, and soft tablets.
